(19) United States
(12) Patent Application Publication
(10) Pub. No.: US 2024/0366842 A1
(43) Pub. Date: Nov. 7, 2024

(54) DECELLULARIZED TISSUE HYDROGELS

(71) Applicant: THE UNIVERSITY OF NOTTINGHAM, Nottingham (GB)

(72) Inventors: Lisa White, Nottingham (GB); Joshua JONES, Nottingham (GB)

(73) Assignee: THE UNIVERSITY OF NOTTINGHAM, Nottingham (GB)

(21) Appl. No.: 18/557,561

(22) PCT Filed: Apr. 29, 2022
(86) PCT No.: PCT/GB2022/051093

§ 371 Date: Oct. 26, 2023

(30) Foreign Application Priority Data
Apr. 30, 2021 (GB) 2106283.1

(51) Int. Cl. A61L 27/52; A61L 27/36

(52) U.S. Cl. CPC A61L 27/52; A61L 27/3604; A61L 27/3608; A61L 27/3629; A61L 27/3687; A61L 2300/80; A61L 2430/40

(57) ABSTRACT

The invention provides a decellularized tissue hydrogel cross-linked with a polyphenol, and a method of preparing a cross-linked decellularized tissue hydrogel, the method comprising the steps of: a) providing at least one decellularized tissue hydrogel; and b) cross-linking the at least one decellularized tissue hydrogel with a polyphenol.

### TECHNICAL FIELD OF THE INVENTION

The present invention relates to decellularized tissue hydrogels cross-linked with a polyphenol; pharmaceutical compositions comprising a decellularized tissue hydrogel cross-linked with a polyphenol; methods of preparing cross-linked decellularized tissue hydrogels; decellularized tissue hydrogels cross-linked with a polyphenol for use in the treatment of a wound, condition or injury; uses of decellularized tissue hydrogels cross-linked with a polyphenol in the manufacture of a medicament for a wound, condition or injury; and methods of medical treatment comprising the use of a decellularized tissue hydrogel cross-linked with a polyphenol.

### BACKGROUND TO THE INVENTION

Mammalian tissues can be decellularized to remove cellular and antigenic material to leave behind the extracellular matrix (ECM). These ECM biomaterials (sometimes termed biologic scaffolds) have been used in millions of patients worldwide in their sheet and powder forms.

Hydrogels include three-dimensional polymeric fibre networks which are hydrophilic and swellable when exposed to water. Hydrogels can be both hydrated or dehydrated. Powdered ECM biomaterials can be solubilised with pepsin and neutralised along with an increase in temperature to generate ECM hydrogels. ECM hydrogels have been shown to be immunomodulatory, pro-regenerative and to promote a constructive re-modelling environment in vivo. The injectable and thermo-responsive nature of these materials also provides additional facility for their use in applications where minimally invasive delivery may be required. Nonetheless, ECM hydrogels have not yet been applied commercially with the first in man safety and feasibility study of a cardiac ECM hydrogel recently completed (Traverse et al JACC: Basic to Translational Science 2019). The soft nature of hydrogels lends well to an array of applications; however, this fragility hinders their application in areas that require stronger materials.

ECM hydrogels may be cross-linked to confer structural strength, rigidity or to delay degradation. Current methods to cross-link ECM materials include the use of glutaraldehyde (GA), EDC-NHS, riboflavin and more recently plant derived agents such as genipin (GP).

GA has been used extensively as a cross-linking agent and has been shown to improve mechanical strength and durability of ECM materials. However, toxicity issues with its use have been reported and it has been associated with calcification leading to deleterious outcomes in vivo (Ma et al. 2014—Crosslinking strategies for preparation of extracellular matrix-derived cardiovascular scaffolds; B Ma, X Wang, C Wu, J Chang; Regenerative Biomaterials; 81-89; (2014)). ECM materials cross-linked with GA have also shown very limited mechanical flexibility; this includes the ability to be compressed or rolled and is a desirable property for potential applications.

Recently, the use of plant derived cross-linking agents has been studied, primarily in attempt to produce cross-linked ECM materials that pose fewer issues surrounding toxicity and calcification. GP is one such cross-linking agent that has been shown to produce cross-linked ECM materials with improved biocompatibility over GA and similar chemical cross-linking agents. Although small increases in mechanical strength and durability have been achieved when employing GP compared with non-cross-linked ECM materials, this increase has been significantly reduced compared to cross-linking with GA.

It would be especially advantageous to be able to produce ECM hydrogels with improved strength and mechanical integrity. GA cross-linking of ECM hydrogels has been trialled and some pre-clinical studies have also recently reported the use of plant-derived agents, such as GP; however, the abovementioned issues experienced with cross-linking ECM materials in general have remained prevalent when using GA and GP.

It is an aim of embodiments of the present invention to provide a decellularized tissue hydrogel, which provides one or more of the following advantages:
Mechanical strength and durability.
Flexibility.
Compressibility; preferably allowing the decellularized tissue hydrogel to be compressed into thin sheets.
Rollability; preferably allowing the decellularized tissue hydrogel to be rolled into various structural forms, such as hollow tubes.
Low toxicity; preferably both in vitro and in vivo.
Biocompatibility.
Inhibition of calcification; preferably both in vitro and in vivo.
Tailored degradation rate.
Pro-regenerative ability.

In particular, it is an aim of embodiments of the invention to provide a cross-linked decellularized tissue hydrogel which provides improved mechanical strength whilst imparting compressibility and/or rollability to the hydrogel, whilst still maintaining ECM structure and function.

It is a further aim of embodiments of the present invention to overcome or mitigate at least one issue of the prior art hereinabove.

It is also an aim of embodiments of the present invention to overcome or mitigate at least one problem of the prior art, whether expressly described herein or not.

### SUMMARY OF THE INVENTION

According to one aspect of the invention, there is provided a decellularized tissue hydrogel cross-linked with a polyphenol.

A “polyphenol” is a compound comprising greater than one phenol unit, wherein each phenol unit may comprise one or more phenolic hydroxyl moieties.

Cross-linking the decellularized tissue hydrogel, with a polyphenol significantly increases its mechanical strength. Decellularized tissue hydrogels cross-linked with a polyphenol are also flexible, compressible, and rollable into various structural forms. The decellularized tissue hydrogel cross-linked with a polyphenol is also biocompatible and non-toxic. It may also possess pro-regenerative ability and allow for inhibition of calcification. Cross-linking the decellularized tissue hydrogel with a polyphenol may also allow for a tailored degradation rate of the decellularized tissue hydrogel.

In some embodiments, the decellularized tissue hydrogel comprises tissue of mammalian origin. In some embodiments, the decellularized tissue hydrogel comprises tissue of bovine, porcine, equine, or human origin.

In some embodiments, the decellularized tissue hydrogel comprises one or more tissues of the group comprising: bone, liver, intestine, spinal cord, placenta, kidney, brain, lung, pancreas, heart, bladder, muscle, rectum, trachea, larynx, pharynx, urethra, gallbladder, spleen, stomach, thyroid, diaphragm, thymus, and nerve. In some embodiments, the decellularized tissue hydrogel comprises placental tissue. Placental tissue may preferably comprise amniotic membrane tissue. The decellularized tissue hydrogel may comprise one or more tissues independently selected from the group comprising: bone, liver, intestine, spinal cord, and amniotic membrane. The decellularized tissue hydrogel may preferably comprise one or more tissues independently selected from the group comprising: bone, liver, intestine, and amniotic membrane. In preferred embodiments, the decellularized tissue hydrogel comprises one or more tissues of the group comprising: bone, liver, and intestine. Such source tissues allow for the formation of a tissue hydrogel with high natural strength and excellent mechanical properties on gelation. Such source tissues are also able to maintain ECM structure and properties and native ECM components post-decellularization, -gelation, and -crosslinking, influencing features such as growth factor binding and cellular response.

In some embodiments, the decellularized tissue hydrogel comprises bone tissue. Such a decellularized tissue hydrogel cross-linked with a polyphenol may provide increased mechanical strength compared with cross-linked decellularized tissue hydrogels obtained from other tissues.

In some embodiments, the decellularized tissue hydrogel comprises bone tissue comprising one or more tissue types of the group comprising: compact tissue, cancellous tissue, subchondral tissue, and combinations of any of the above tissue types.

In some embodiments, the decellularized tissue hydrogel comprises bone tissue from tibia, fibula, femur, pelvic girdle, ribs, vertebral column, skull, scapula, humerus, radius, or ulna.

In some embodiments, the polyphenol comprises one or more compounds of the group comprising: a flavonoid, a hydroxybenzoic acid, a polyphenolic amide, a hydroxycinnamic acid, a stilbene, a lignan, and combinations thereof.

In some embodiments, the polyphenol comprises a flavonoid comprising a condensed tannin.

In some embodiments, the polyphenol comprises a condensed tannin comprising a proanthocyanidin or any glycoside thereof. Such proanthocyanidin polyphenols may display improved cross-linking ability, provide tissue with greater mechanical strength, compressibility and/or rollability, and/or be particularly effective at retaining the natural architecture of the ECM of the tissue, compared to other cross-linking agents (including other polyphenols).

In some embodiments, the polyphenol comprises a proanthocyanidin comprising one or more compounds of the group comprising: a procyanidin, a propelargonidin, a prodelphinidin, a profisetinidin, a proteracacinidin, a proguibourtinidin, a prorobinetidin, a propetunidin, a promalvidin, a propeonidin, and combinations thereof.

In some preferred embodiments, the polyphenol comprises an oligomeric procyanidin formed from catechin and epicatechin.

In some embodiments, the polyphenol is of plant origin, which may comprise fruit, nut, vegetable, or spice.

In some embodiments, the polyphenol is of nut origin, the nut comprising one or more of the group comprising: hazelnut, chestnut, flax seed, walnut, almond, pecan, and combinations thereof. In some embodiments, the polyphenol is of vegetable origin, the vegetable comprising one or more of the group comprising: artichoke, chicory, onion, spinach, asparagus, broccoli, carrot, endive, potato, lettuce, shallot, legumes, and combinations thereof. In some embodiments, the polyphenol is of spice origin, the spice comprising one or more of the group comprising: caraway, celery seed, cinnamon, turmeric, clove, cumin, curry powder, basil, marjoram, parsley, peppermint, spearmint, lemon verbena, oregano, rosemary, sage, star anise, thyme, and combinations thereof. In some embodiments, the polyphenol is of fruit origin, the fruit comprising one or more of the group comprising: grape, apple, aronia fruit, bilberry, cranberry, blackcurrant, fruit of the açai palm, elderberry, strawberry, blueberry, blackberry, marion berry, choke berry, raspberry, cherry, peach, pear, nectarine, plum, apricot, kiwi, avocado, mango, date, banana, grapefruit, pomegranate, and combinations thereof.

In some embodiments, the polyphenol is of grape origin. The polyphenol may be of grape seed or grape skin origin. Such sources are rich in polyphenols, and especially proanthocyanidins. Such sources are also rich in antioxidants.

The polyphenol may be from a plant independently selected from: a shrub, and a tree. The polyphenol may be from an evergreen shrub, preferably from a tea shrub. The polyphenol may be of plant leaf origin, preferably of tea leaf origin. The polyphenol may be from a Camelia sinensis shrub and/or from a Camelia taliensis shrub, preferably from leaves thereof. The polyphenol may from a Camelia sinensis var. sinensis shrub and/or from a Camelia sinensis var. assamica shrub. The polyphenol may preferably be of tea origin. The polyphenol may be of tea origin, said tea being independently selected from the group comprising: white tea, yellow tea, green tea, oolong tea, dark tea, black tea, and combinations thereof. The polyphenol may alternatively be of twig tea origin.

The polyphenol of tea origin may comprise at least one catechin and/or at least one flavonol. The polyphenol may comprise both at least one catechin and at least one flavonol, and may have a catechin to flavonol ratio of at least 1:1, or at least 2:1, 3:1, 4:1, 5:1, 6:1, 7:1, 8:1, or at least 9:1. The polyphenol may comprise at least one catechin independently selected from the group comprising: catechin, epicatechin, epigallocatechin, epicatechin gallate, epigallocatechin gallate, and combinations thereof. The polyphenol may comprise at least one flavonol independently selected from the group comprising: kaempferol, quercitin, a myricetin glycoside, and combinations thereof.

In some embodiments, the decellularized tissue hydrogel is at least 5, 10, 15, 20, 25, 30, 35, 40, 45, 50, 55, 60, 65, 70, 75, 80. 85, 90, 91, 92, 93, 94, 95, 96, 97, 98, 99, or at least 99.5% or substantially fully cross-linked with a polyphenol. The cross-linking increases the mechanical properties of the resultant hydrogel, including resistance to degradation.

In some embodiments, only a part of the decellularized tissue hydrogel is crosslinked with a polyphenol. In other embodiments, the entire tissue hydrogel is crosslinked with a polyphenol. In some embodiments, at least 1% by volume of the decellularized tissue hydrogel is crosslinked with a polyphenol, or at least 2, 3, 4, 5, 6, 7, 8, 9, 10, 15, 20, 25, 30, 35, 40, 45, 50, 55, 60, 65, 70, 75, 80, 85, 90, or at least 95% by volume of the decellularized tissue hydrogel is crosslinked. In some embodiments, no greater than 95% by volume of the decellularized tissue is crosslinked with a polyphenol, or no greater than 90, 85, 80, 75, 70, 65, 60, 55, 50, 45, 40, 35, 30, 25, 20, 15, 10, or no greater than 5% by volume of the decellularized tissue hydrogel is crosslinked. In some embodiments, between 5-95% by volume of the decellularized tissue hydrogel is crosslinked with a polyphenol, or between 10-90, 20-80, 30-70, 40-60, or between 5-90, 5-80, 5-70, 5-60, 5-50, 5-40, 5-30, 5-20, 5-10, or between 10-95, 20-95, 30-95, 40-95, 50-95, 60-95, 70-95, 80-95, or between 90-95% by volume of the decellularized tissue hydrogel is crosslinked with a polyphenol.

In some embodiments, the decellularized tissue hydrogel is crosslinked with a polyphenol at and/or on an outer surface thereof. The hydrogel may only be crosslinked with a polyphenol at and/or on its outer surface. In some embodiments, the hydrogel is crosslinked with a polyphenol at and/or on at least 5% of the outer surface of the decellularized tissue hydrogel, or at least 10, 20, 30, 40, 50, 60, 70, 80, or at least 90%, or substantially 100% of the outer surface area of the tissue hydrogel. In some embodiments, the hydrogel is crosslinked with a polyphenol at and/or on no greater than 95%, or no greater than 90, 80, 70, 60, 50, 40, 30, 20, 10, or no greater than 5% of the outer surface area of the tissue hydrogel. In some embodiments, the hydrogel is crosslinked with a polyphenol at and/or on between 5-95% of the outer surface area of the tissue hydrogel, or between 10-90, 20-80, 30-70, 40-60, or between 5-90, 5-80, 5-70, 5-60, 5-50, 5-40, 5-30, 5-20, 5-10, or between 10-95, 20-95, 30-95, 40-95, 50-95, 60-95, 70-95, 80-95, or between 90-95% of the outer surface area of the tissue hydrogel.

Selectively crosslinking different parts of the tissue hydrogel and to varying degrees allows for a final gel having different mechanical properties on different parts thereof. This allows for the simple construction of final crosslinked products with complex mechanical properties which can be tailored based on their proposed uses.

In some embodiments, the decellularized tissue hydrogel comprises sulphated glycosaminoglycans (sGAGs). The presence of sGAGs may influence features such as growth factor binding and cellular response.

The decellularized tissue hydrogel cross-linked with a polyphenol may possess strong modulatory properties, providing cells with adhesion sites and biological factors, such as growth factors and cryptic peptides.

According to a second aspect of the invention, there is provided a pharmaceutical composition comprising a decellularized tissue hydrogel cross-linked with a polyphenol.

In some embodiments, the pharmaceutical composition is in an injectable form, which may comprise an injectable liquid or gel.

In some embodiments, the pharmaceutical composition is in the form of a topical composition. The topical composition may comprise a paste, gel, sheet, membrane, or patch The composition may be applied to heart tissue and may take the form of a pericardial patch.

In some embodiments, the pharmaceutical composition is in the form of a tissue wrap. The tissue wrap may comprise a sheet or conduit. The tissue wrap may comprise a nerve wrap or a tendon wrap.

In some embodiments, the pharmaceutical composition is in the form of an implant. The implant may comprise a sensory or neurological implant, a cosmetic implant, or an organ or tissue implant. The organ or tissue implant may comprise a cardiovascular implant, a gastrointestinal implant, a respiratory implant, a urological implant, a dermal implant or preferably an orthopaedic implant, which may comprise a bone implant.

In some embodiments, the decellularized tissue hydrogel cross-linked with a polyphenol comprises at least 0.5, 1, 2, 3, 4, 5, 6, 7, 8, 9, 10, 15, 20, 25, 30, 35, 40, 45, 50, 55, 60, 65, 70, 75, 80, 85, 90, 95%, or up to 100% by weight of the pharmaceutical composition.

The decellularized tissue hydrogel cross-linked with a polyphenol may be in accordance with any decellularized tissue hydrogel cross-linked with a polyphenol of the first aspect of the invention.

According to a third aspect of the invention, there is provided a method of preparing a cross-linked decellularized tissue hydrogel, the method comprising the steps of:
a. Providing at least one decellularized tissue hydrogel; and
b. Cross-linking the at least one decellularized tissue hydrogel with a polyphenol.

In some embodiments, step (a) comprises decellularizing tissue to provide decellularized tissue; digesting the decellularized tissue with at least one enzyme to form a digest; and neutralising the digest.

In some embodiments, step (a) of the method comprises preparing the at least one decellularized tissue hydrogel from bone tissue by a method comprising the steps of: optionally demineralising and delipidating the bone tissue, decellularizing the bone tissue, digesting the decellularized bone tissue with at least one enzyme to form a digest, and neutralising the digest.

The step of demineralising the bone tissue may comprise subjecting the bone tissue to hydrochloric acid. This step may be performed for at least 24 hours. The bone tissue may be broken into fragments prior to this step.

The step of delipidating the bone tissue may comprise subjecting the bone tissue to a mixture of chloroform and methanol. This step may be performed for at least 1 hour. The chloroform and methanol may be used as a 1:1 mixture.

Step (a) may comprise a step of decellularizing a tissue by subjecting the tissue, preferably bone tissue to an enzyme. The enzyme may comprise a proteolytic enzyme, which may comprise an endopeptidase. The endopeptidase may comprise chymotrypsin, elastase, thermolysin, glutamyl endopeptidase, neprilysin, papain, pepsin, or trypsin. The enzyme may be a nuclease, which may be selected from a DNase and RNase. In preferred embodiments the decellularization step may comprise subjecting the tissue, preferably bone tissue to at least one proteolytic enzyme (preferably trypsin).

The enzyme may be coupled with a chelating agent. The chelating agent may comprise a multidentate ligand, such as ethylenediamine, acetylacetonate, phenanthroline, oxalate, a crown ether, methyl Tm ligand, trispyrazolylborate, tris(4,4-dimethyl-2-oxazolinyl) phenyl borate, tris(4S-isopropyl-2-oxazolinyl) phenyl borate, N,N,N′,N″,N″-pentamethyldiethylenetriamine, bis(diphenylphosphinoethyl)phenylphosphine, 1,4, 7-trithiacyclononane, 1,4,7-trimethyl-1,4,7-triazacyclononane, cis,cis-1,3,5-triaminocyclohexane, chlorin, corrin, 1,4,8,11-tetraazacyclotetradecane, 1,4,7,10-tetraazacyclododecane, Dibenzotetramethyltetraaza[14]annulene, N,N-ethylenediaminediacetic acid, N,N′-ethylenediaminediacetic acid, N-hydroxyimino-2,2′-dipropionic acid, diethylenetriamineacetic acid, iso-diethylenetriamineacetic acid, Jäger's N2O2 ligand, naphthalocyanine, nitrilotriacetic acid, phthalocyanine, porphyrin, rhodotorulic acid, salen, salpn, tetraphos, 1,4,7,10-tetrathiadodecane, 1,4,7,10-tetrathiatridecane, 1,4,8,11-tetrathiatetradecane, 1,4,8,12-tetrathiapentadecane, 1,5,9,13-tetrathiahexadecane, 2,5,8-trithia[9](2,5)thiophenophane, Triethylene glycol dimethyl ether, Triethylenetetramine, tris-(o-diphenylphosphinophenyl)-phosphine, Tris(2-pyridylmethyl)amine, 2,2′-bi-1,10-phenanthroline, heme, ethylenediaminetriactetic acid, N,N,N′,N′-tetrakis(2-pyridinylmethyl)-1,2-ethanediamine, DOTA, DTPA, or ethylenediaminetetraactetate (EDTA).

In some embodiments, the enzyme comprises trypsin coupled with EDTA.

In some embodiments decellularization may comprise subjecting the tissue, preferably bone tissue to a detergent treatment. Suitable detergents include SDS, sodium deoxycholate, Triton X-100 or the like, for example. Decellularization may comprises subjecting the tissue, preferably bone tissue to at least one detergent and at least one enzyme, as described above (especially both a protease and a nuclease, preferably in combination with SDS and/or Triton X-100).

In some embodiments, decellularization may comprise subjecting the tissue to treatment with at least one peroxide. The peroxide may comprise a peracid, preferably peracetic acid. Decellularization may comprise subjecting the tissue to both a detergent treatment and treatment with at least one peroxide, which may be performed separately or simultaneously.

In other embodiments, the decellularization may comprise an osmotic shock treatment, freeze-thawing, mechanical decellularization or a combination thereof, which may also be combined with a detergent and/or enzyme treatment.

Decellularized tissue, preferably bone tissue may be freeze-dried and optionally stored at a temperature of −20° C. or lower prior to digesting with the at least one enzyme.

The at least one enzyme used for the digestion step may comprise a proteolytic enzyme, which may comprise an endopeptidase. The endopeptidase may comprise chymotrypsin, elastase, thermolysin, glutamyl endopeptidase, neprilysin, papain, trypsin, or pepsin.

The decellularized tissue, preferably bone tissue may be digested with the at least one enzyme for at least 1 or 2 days, or at least 3, 4, 5, or 6 days.

The decellularized tissue, preferably bone tissue may be subjected to agitation when digested with at least one enzyme.

The digest may be stored at a temperature of −20° C. or lower prior to neutralising the digest.

The digest may be neutralised with neutralisation buffer comprising phosphate-buffered saline (PBS) and sodium hydroxide.

In some embodiments, step (a) of the method further comprises moulding the at least one decellularized tissue hydrogel.

The at least one decellularized tissue hydrogel may be moulded by incubation in a mould for at least 10, 20, 30, 40, 50 minutes, 60 minutes, 70 minutes, 80 minutes, 90 minutes, 100 minutes, 110 minutes or at least 120 minutes.

The at least one decellularized tissue hydrogel may be moulded by incubation in a mould at a temperature of 37° C., which may be in the presence or absence of carbon dioxide (e.g., moulded in an oven or a cell/tissue culture incubator).

In some embodiments, step (b) of the method comprises cross-linking the at least one decellularized tissue hydrogel with a polyphenol for at least 10, 20, 30, 40, 50 minutes, or at least 1 hour, 1 hour 10, 20, 30, 40, 50 minutes, or at least 2, 4, 6, 8, 10, 12, 14, 16, 18, 20, 22, 24, 26, 28, 30, 32, 34, 36, 38, 40, 42, 44, 46, 48, or at least 50 hours.

In some embodiments, step (b) of the method further comprises subjecting the at least one decellularized tissue hydrogel to agitation. This may be achieved using an orbital shaker.

In some embodiments, the polyphenol has a concentration of at least 0.1, 0.2, 0.3, 0.4, 0.5, 0.6, 0.7, 0.8, 0.9 mM, or at least 1, 2, 3, 4, 5, 6, 7, 8, 9, or at least 10, 15, 20, 25, 30, 35, 40, 45, 50, 55, 60, 65, 70, 75, 80, 85, 90, 95, or at least 100 mM. In embodiments wherein the polyphenol is or comprises procyanidin (PA), the polyphenol preferably has a concentration of at least 1 mM.

In some embodiments, the polyphenol has a concentration of no greater than 200 mM, or no greater than 180, 160, 140, 120, 100, 90, 80, 70, 60, or preferably of no greater than 50 mM, or of no greater than 45, 40, 35, 30, 25, 20, or of no greater than 15 mM.

The polyphenol may have a concentration of between 0.1-50 mM, or of between 0.25-40, 0.5-30, 0.75-20, or preferably of between 1-10 mM. Such concentrations allow for a minimised risk of degradation and provide a cross-linked hydrogel with excellent rheological properties.

In some embodiments, the polyphenol is dissolved in a solvent, which may comprise an aqueous solvent. The aqueous solvent may comprise water, which may comprise a dissolved salt. The aqueous solvent may comprise water and an organic solvent, which may comprise a polar organic solvent. The polar organic solvent may comprise a polar protic solvent, which may comprise a carboxylic acid, such as formic acid or acetic acid; or an alcohol, such as methanol, ethanol, n-propanol, isopropyl alcohol, or n-butanol. The polar organic solvent may comprise a polar aprotic solvent, which may comprise a carbonate, such as propylene carbonate; a nitrile, such as acetonitrile; an amide, such as dimethylformamide; a ketone, such as acetone; an ester, such as ethyl acetate; an organohalide, such as dichloromethane or chloroform; an ether, such as tetrahydrofuran, diethyl ether, or 1,4-dioxane; or preferably, a sulphoxide, such as dimethylsulphoxide (DMSO). The solution may comprise an organic solvent concentration of at least 0.5, 1, 2, 3, 4, 5, 6, 7, 8, 9%, or at least 10, 15, 20, 25, 30, 35, or at least 40% v/v. The solution may comprise an organic solvent concentration of no greater than 50% v/v, or no greater than 40, 30, 20, or no greater than 10% v/v. The solution may comprise an organic solvent concentration of between 0.5-25% v/v, or between 1-20, 2.5-15, or of between 5-10% v/v.

In some embodiments, the polyphenol has a concentration of between 0.1-50 mM, and is dissolved in a solvent having an organic solvent concentration of between 0.5-25% v/v, or between 1-20, 2.5-15, or between 5-10% v/v. The polyphenol may have a concentration of between 0.25-40 mM, and may be dissolved in a solvent having an organic solvent concentration of between 0.5-25% v/v, or between 1-20, 2.5-15, or between 5-10% v/v. The polyphenol may have a concentration of between 0.5-30 mM, and may be dissolved in a solvent having an organic solvent concentration of between 0.5-25% v/v, or between 1-20, 2.5-15, or between 5-10% v/v. The polyphenol may have a concentration of between 0.75-20 mM, and may be dissolved in a solvent having an organic solvent concentration of between 0.5-25% v/v, or between 1-20, 2.5-15, or between 5-10% v/v. The polyphenol may preferably have a concentration of between 1-10 mM, and may be dissolved in a solvent having an organic solvent concentration of between 0.5-25% v/v, or between 1-20, 2.5-15, or between 5-10% v/v.

In preferred embodiments, the solvent is or comprises a polar aprotic solvent. The polar aprotic solvent may preferably comprise a carbonate and/or a sulfoxide. The carbonate may preferably be or comprise an organic carbonate. The carbonate may be or comprise a C1-C15 carbonate, C1-C10 carbonate, or a C1-C5 carbonate. In a particularly preferred embodiment, the carbonate is or comprises propylene carbonate. The sulfoxide may preferably be or comprise dimethyl sulfoxide.

In some embodiments, the polyphenol is dissolved in a solvent comprising a pluronic (poloxamer). The pluronic may comprise a triblock copolymer comprising a central hydrophobic chain flanked by two hydrophilic chains. The hydrophobic chain may comprise polyoxypropylene or a derivative thereof. The hydrophilic chains may comprise polyoxyethylene or a derivative thereof. The pluronic may comprise Pluronic F127.

In embodiments wherein the polyphenol is obtained from a shrub or tree, preferably wherein the polyphenol is obtained from tea, the method may comprise dissolving the polyphenol in water. The polyphenol may be dissolved by submerging a plant part, preferably tea leaves in the water. The water may have a temperature of at least 25° C., or at least 30, 40, 50, or at least 60° C., or at least 70, 80, 90, or at least 95° C. The plant part may be submerged in the water for at least 5 minutes, or at least 10, 20, 30, 40, 50, or at least 60 minutes. The plant part may be submerged in the water for between 10-110 minutes, or between 20-100, 30-90, 40-80, or between 50-70 minutes.

In some embodiments, step (b) further comprises washing the cross-linked decellularized tissue hydrogel with PBS and/or distilled water.

The cross-linked decellularized tissue hydrogel prepared by way of the method of the third aspect of the invention may be in accordance with any decellularized tissue hydrogel cross-linked with a polyphenol of the first and second aspects of the invention.

According to a fourth aspect of the invention, there is provided a decellularized tissue hydrogel cross-linked with a polyphenol for use in the treatment of a wound, condition or injury.

The wound, condition or injury may comprise a wound comprising a lesion, a tear, a cut, an incised wound, a laceration, an abrasion, a puncture, an avulsion, an amputation, a penetration wound, a gunshot wound, a hematoma, a crush wound, or a critical wound. The wound, condition or injury may comprise a condition comprising a physical or genetic condition, which may comprise a blood condition, a cancer, a neoplasm, a cardiovascular condition, a congenital disorder, an ear condition, an eye condition, an infection, an inflammatory condition, an immune system condition, a metabolic condition, a neurological condition, an oral condition, a gastrointestinal condition, a renal condition, a urogenital condition, a skin condition, an adhesion, or a musculoskeletal condition. The wound, condition or injury may comprise an internal or external injury comprising a traumatic injury, a bruise, a burn, a dislocation, an electrical injury, a fracture, a sprain, internal bleeding, a catastrophic injury, a toxin injury, a radiation-induced injury, a frostbite injury, a skin injury, a head injury, an eye injury, a brain injury, a nerve injury, a soft tissue injury, a musculoskeletal injury, an organ injury, a foot injury, a knee injury, a back injury, a hand injury, or a chest injury. The injury may comprise an accidental or intentional injury, which may comprise an injury due to an act of violence, an injury due to self-harm, a vehicle injury, a sports injury, a climbing injury, a bite, or an occupational injury.

The treatment may comprise replacing damaged or removed tissue, promoting repair and regeneration of damaged or removed tissue, and/or modulating inflammation

In some embodiments, the wound, condition or injury comprises a wound, condition or injury to bone, nerves, tendons, or abdominal regions. The wound, condition or injury to abdominal regions may comprise a wound, condition or injury to intestine, which may comprise a colorectal cancer, a colonic polyp, an ulcerative colitis, a diverticulitis, an intestinal obstruction, or preferably a peritoneal adhesion. The wound, condition or injury to nerves may comprise a tumour, neurofibromatosis, a scarred nerve, or a damaged nerve, preferably a damaged peripheral nerve. The wound, condition or injury to bone may comprise damaged bone, which may comprise an open fracture, a closed fracture, a partial fracture, a complete fracture, a stable fracture, a displaced fracture, a transverse fracture, a spiral fracture, a greenstick fracture, a stress fracture, a compression fracture, an oblique fracture, an impacted fracture, a segmental fracture, a comminuted fracture, or an avulsion fracture.

Damaged bone may comprise a non-union fracture and/or a critical size defect (non-healing defect).

The decellularized tissue hydrogel cross-linked with a polyphenol may be in accordance with any decellularized tissue hydrogel cross-linked with a polyphenol of the first, second, and third aspects of the invention.

According to a fifth aspect of the invention, there is provided the use of a decellularized tissue hydrogel cross-linked with a polyphenol in the manufacture of a medicament for a wound, condition or injury.

The wound, condition or injury may be in accordance with any wound, condition or injury of the fourth aspect of the invention.

The decellularized tissue hydrogel cross-linked with a polyphenol may be in accordance with any decellularized tissue hydrogel cross-linked with a polyphenol of the first, second, third, and fourth aspects of the invention.

The method of producing the decellularized tissue hydrogel cross-linked with a polyphenol may be in accordance with any method of the third aspect of the invention. According to a sixth aspect of the invention, there is provided a method of treating a subject in need of treatment with a decellularized tissue hydrogel cross-linked with a polyphenol.

In some embodiments, the method may be a method of medical or surgical treatment, which may comprise elective treatment, semi-elective treatment, or emergency treatment. The method of medical or surgical treatment may comprise exploratory treatment, therapeutic treatment, or cosmetic treatment. The method of medical or surgical treatment may comprise amputation, resection, segmental resection, excision, extirpation, replantation, reconstructive surgery, transplant surgery, organ or tissue replacement or removal. The method of medical or surgical treatment may comprise minimally-invasive treatment or an open surgical procedure. The method of medical or surgical treatment may comprise laser surgery, microsurgery, or robotic surgery.

In some embodiments, the method is used to treat a wound, condition or injury.

The wound, condition or injury may be in accordance with any wound, condition or injury of the fourth and fifth aspects of the invention.

The method may comprise applying the decellularized tissue hydrogel cross-linked with a polyphenol to a wound, replacing damaged tissue with the decellularized tissue hydrogel cross-linked with a polyphenol, or injecting the decellularized tissue hydrogel cross-linked with a polyphenol into the subject.

In some embodiments, the method may comprise administering a decellularized tissue hydrogel to the subject and cross-linking the decellularized tissue hydrogel with a polyphenol to form the decellularized tissue hydrogel cross-linked with a polyphenol in situ. The decellularized tissue hydrogel may take the form of a fluid and may be injected into the subject. The polyphenol may take the form of a fluid and may be injected into the subject. The polyphenol and decellularized tissue hydrogel may be administered or implanted together, or the decellularized tissue hydrogel may be implanted initially, followed by subsequent addition of the polyphenol.

In other embodiments the method may comprise implanting a cross-linked decellularized tissue hydrogel into the subject.

The decellularized tissue hydrogel cross-linked with a polyphenol may be in accordance with any decellularized tissue hydrogel cross-linked with a polyphenol of the first, second, third, fourth, and fifth aspects of the invention.

### DETAILED DESCRIPTION OF THE INVENTION

In order that the invention may be more clearly understood embodiments thereof will now be described, by way of example only, with reference to the accompanying drawings, of which:

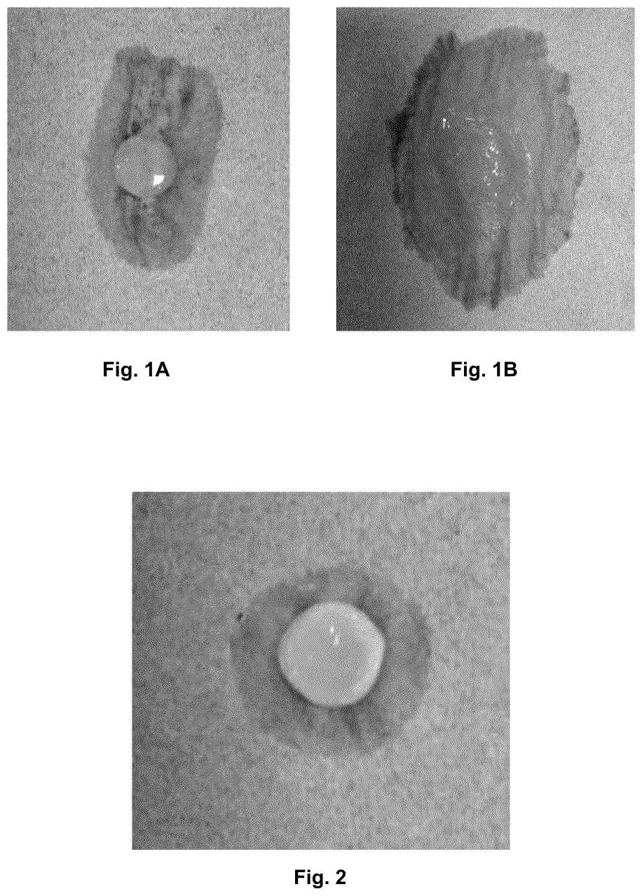

FIGS. 1A and 1B show non cross-linked decellularized bone tissue hydrogels.

FIG. 1A shows the decellularized bone tissue hydrogel as formed and FIG. 1B shows the decellularized bone tissue hydrogel when compressed.

FIG. 2 shows a GA cross-linked decellularized bone tissue hydrogel when compressed.

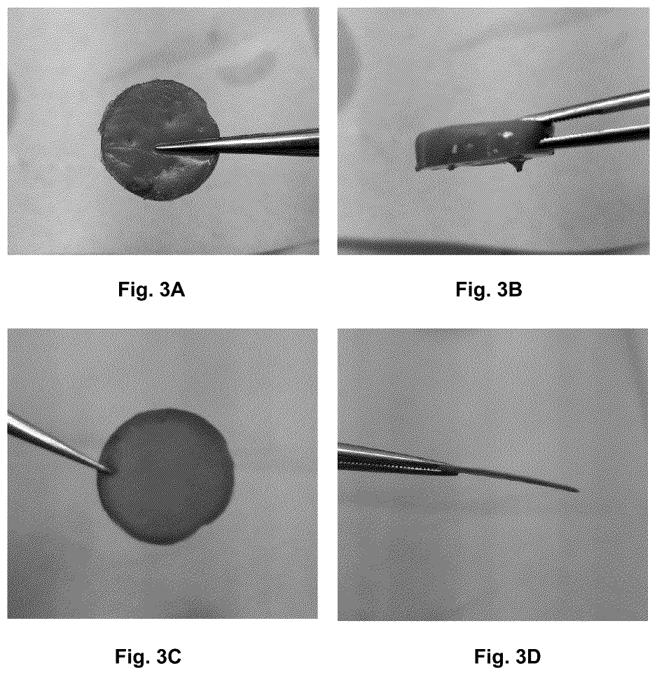

FIG. 3A to 3D show decellularized bone tissue hydrogels cross-linked with a polyphenol (procyanidin (PA)). FIGS. 3A and 3B show PA cross-linked decellularized bone tissue hydrogel as formed and FIGS. 3C and 3D show PA cross-linked decellularized bone tissue hydrogel when compressed into a flat sheet.

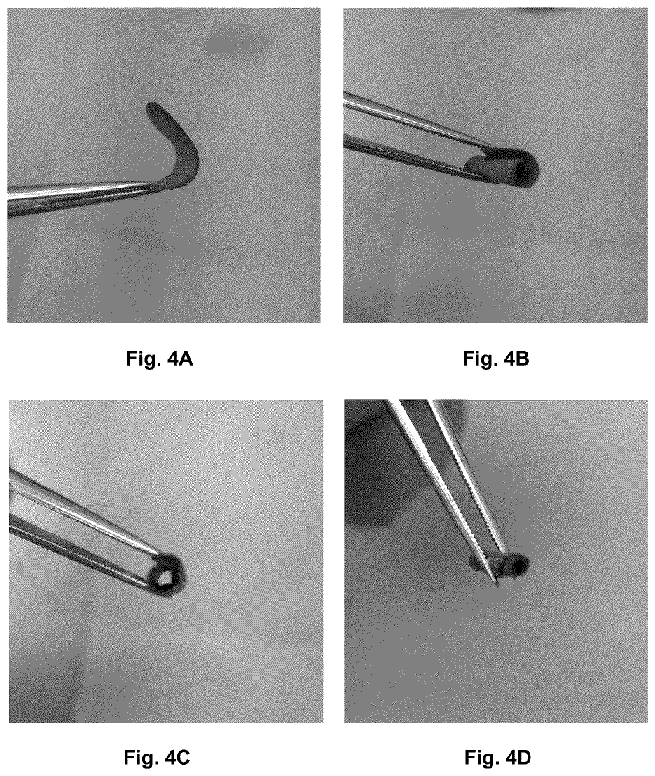

FIG. 4A to 4D show PA cross-linked decellularized bone tissue hydrogels being rolled into hollow tubes. FIG. 4A shows a PA cross-linked decellularized bone tissue hydrogel in a curled formation, FIGS. 4B and 4C show the PA cross-linked decellularized bone tissue hydrogel loosely rolled to form a hollow tube and FIG. 4D shows the PA cross-linked decellularized bone tissue hydrogel tightly rolled to form another hollow tube.

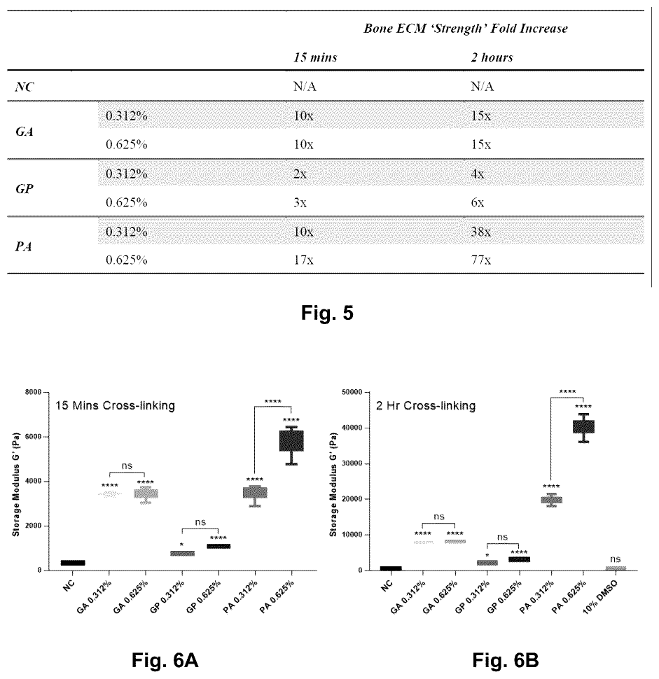

FIG. 5 provides an overview of strength increases upon cross-linking of a decellularized bone tissue hydrogel with GA, GP, and PA relative to a non-cross-linked decellularized bone tissue hydrogel (NC).

FIG. 6A and 6B show storage moduli of non cross-linked (NC), GA cross-linked, GP cross-linked, and PA cross-linked decellularized bone tissue hydrogels. FIG. 6A shows storage moduli achieved with 15 minutes cross-linking and FIG. 6B shows storage moduli achieved with 2 hours cross-linking.

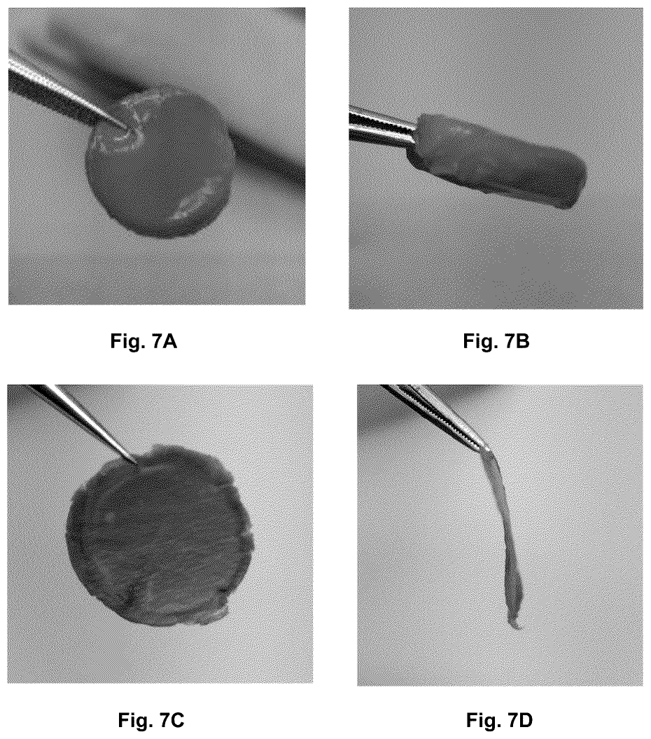

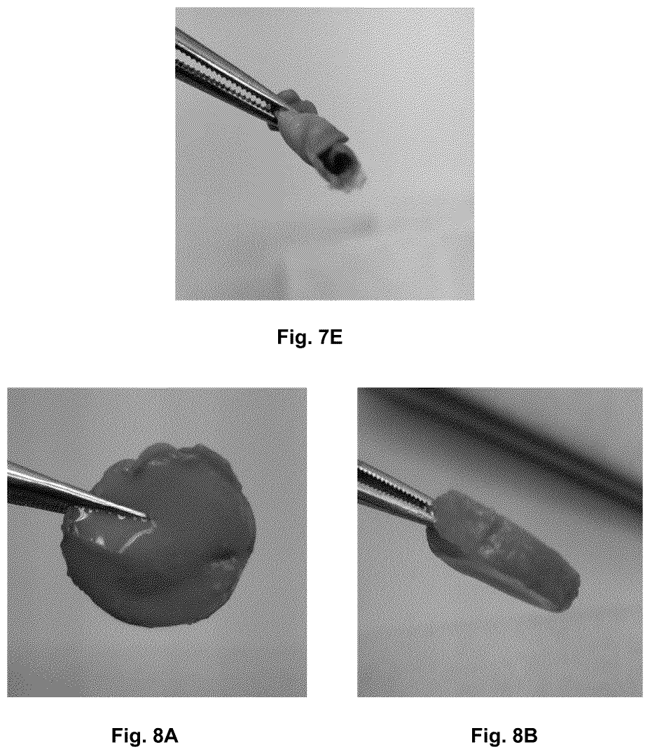

FIG. 7A to 7E show PA cross-linked decellularized liver tissue hydrogels. FIGS. 7A and 7B show the PA cross-linked decellularized liver tissue hydrogels as formed, FIGS. 7C and 7D show the PA cross-linked decellularized liver tissue hydrogels when compressed into flat sheets, and FIG. 7E shows a decellularized liver tissue hydrogel when rolled to form a hollow tube.

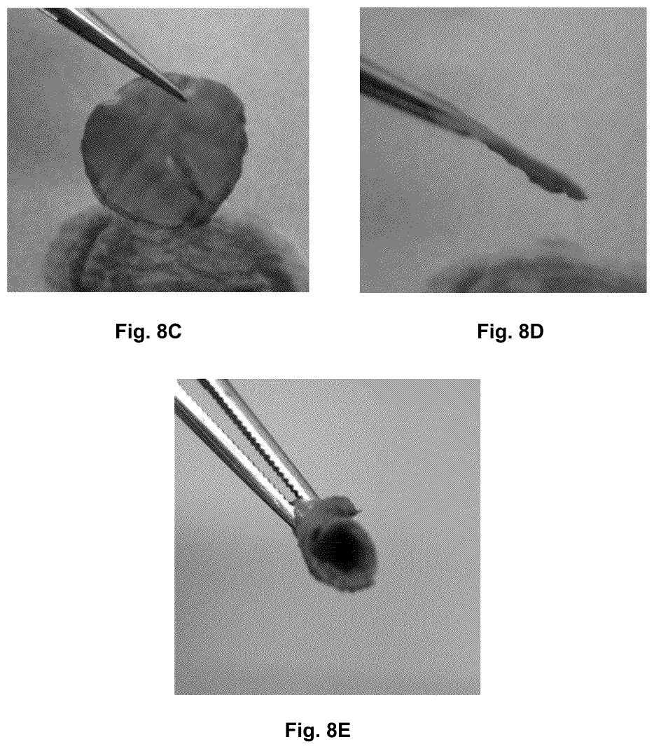

FIG. 8A to 8E show PA cross-linked decellularized small intestine submucosa (SIS) tissue hydrogels. FIGS. 8A and 8B show the PA cross-linked decellularized SIS tissue hydrogels as formed, FIGS. 8C and 8D show the PA cross-linked decellularized SIS tissue hydrogels when compressed into flat sheets, and FIG. 8E shows a decellularized SIS tissue hydrogel when rolled to form a hollow tube.

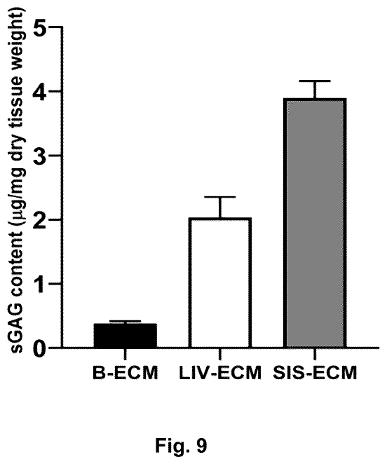

FIG. 9 shows sulphated glycosaminoglycan (sGAG) contents of non-crosslinked decellularized bone, liver and SIS tissue hydrogels.

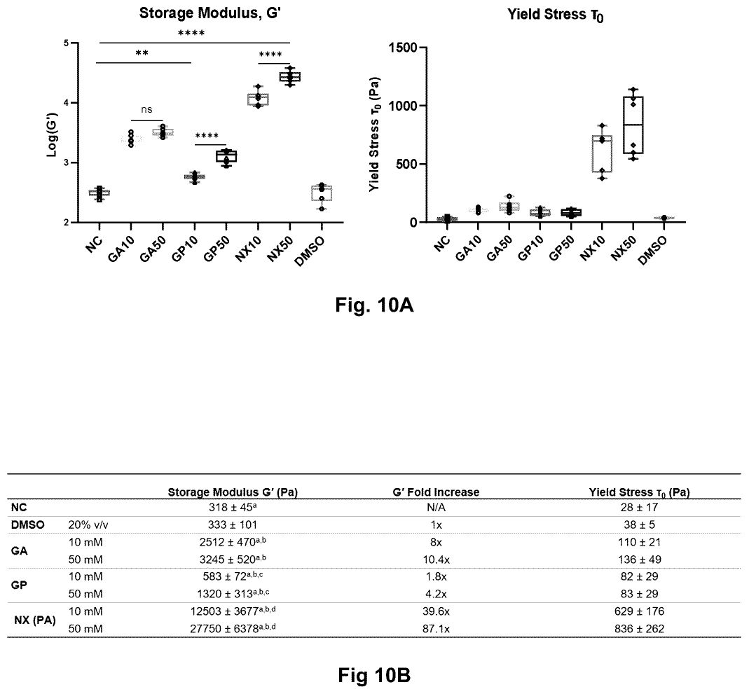

FIGS. 10A and 10B show storage moduli and yield stress of different concentrations of non cross-linked (NC), GA cross-linked, GP cross-linked, and PA (NX) cross-linked decellularized bone tissue hydrogels

### EXAMPLES

All experiments were approved by a local ethical review committee and carried out under UK Home Office approval and according to the Animals Scientific Procedures Act (1986).

### Example 1—Preparation of a PA Cross-Linked Decellularized Bone Tissue Hydrogel of the Invention

A first embodiment of a PA cross-linked decellularized tissue hydrogel according to the invention was prepared by the following process.

### Tissue Processing and Decellularization

Fresh bovine tibiae were obtained through an EU certified butcher (J. Broomhall Ltd Butchers Shop, Dursley, Gloucestershire, UK), with cancellous bone separated and used for processing. Demineralisation, delipidation and decellularization of the bone tissue was thereafter performed based on the protocol outlined by Sawkins et al (M. J. Sawkins, W. Bowen, P. Dhadda, H Markides, L. E. Sidney, A. J. Taylor, F. R. A. J. Rose, S. Badylak, K. M. Shakesheff, L. J. White, Hydrogels derived from demineralized and decellularized bone extracellular matrix, Acta Biomaterialia. 9 (2013), 7865-7873).

Firstly, the bone tissue was immersed in a 0.1% w/v solution of gentamicin for 1 hour, followed by five washes in sterile deionised water (dH₂O). Bone tissue was frozen with liquid nitrogen before breaking into small fragments using a hammer, with the cortical bone separated from the sample and discarded. The remaining fragmented cancellous bone was frozen before ground into smaller fragments in a coffee grinder.

Demineralisation occurred through immersion of cancellous bone fragments in 0.5 M hydrochloric acid (HCl) at a volume of 25 mL per gram of bone tissue. The solution underwent constant agitation on a magnetic stirrer at 300 rpm for 24 hours and was then washed in dH₂O at a volume five times that of HCl. Demineralised bone was then air dried and blotted to remove excess moisture.

Demineralised bone then had lipids removed through a modified Bligh and Dyer approach (E. G. Bligh, W. J. Dyer, A rapid method of total lipid extraction and purification, Canadian Journal of Biochemistry and Physiology 37(8)(1959) 911-917). Chloroform and methanol were mixed at a 1:1 ratio and used at a volume of 30 mL per gram of demineralised bone tissue. The sample was immersed in this solution for 1 hour under constant agitation at 300 rpm, then washed in methanol at two times volume, then phosphate buffered saline (PBS) at five times volume. The resultant sample is now referred to as demineralised bone matrix (DBM), and was lyophilised prior to decellularization.

Decellularization was enzymatically based using a 0.05% (w/v) trypsin and 0.02% (w/v) ethylenediaminetetraacetic acid (EDTA). The DBM was immersed in the trypsin/EDTA solution at 30 mL per gram of tissue and stirred at 300 rpm for 24 hours, then the trypsin/EDTA solution was replaced with fresh PBS and stirred for another 24 hours. Following this, the resulting decellularized bone ECM (bECM) was lyophilised and stored at −20° C. until use.

Quantity of DNA was used as a metric to assess decellularization success. The bECM powder was first digested with proteinase K in lysis buffer (10 mM Tris-HCl, 100 mM NaCl, 10 mM EDTA, 0.5% SDS, 5 ul/ml proteinase K) before extracting DNA with a standard phenol-chloroform-isoamyl process. Extracted double stranded DNA (dsDNA) was quantified using the picogreen assay kit (Invitrogen Ltd, Paisley, UK) as per the supplied protocol. The amount of DNA significantly decreased (unpaired T-test, p<0.0001, n=3) between the native and decellularized bone tissue (939.3±3.197 ng/mg and 21.46±0.7958 ng/mg respectively). The DNA content of the decellularized bone tissue was <50 ng/mg dry weight signalling a successful decellularization.

Sulphated glycosaminoglycans (sGAG) content of the bECM powder was assessed using a dimethylmethylene blue (DMMB) colourimetric assay. A 1 ml volume of papain solution (100 mM Na₂HPO₄, 10 mM EDTA, 10 mM L-cysteine, 125 μg/ml papain enzyme) was added to 10-50 mg of bECM powder and digested at 65° C. until fully solubilised. The 1 mL papain solution could have been added to 10-50 mg of bECM powder, as the results are normalised to the initial dry weight. The sGAG content was then assessed by adding 20 μl of sample to 200 μl of DMMB reagent (16 mg/L DMMB, 40 mM glycine, 40 mM NaCl, taken to pH 1.5 using appropriate amount of HCl) and measuring the difference in the absorbance readings between 525 nm and 595 nm. A standard curve was generated using known concentrations of shark chondroitin sulphate in the papain solution, to which the sample were compared against. Using the DMMB assay allowed for the quantification of sGAGs in the bECM powder, which was determined to be 53.38±4.391 ng/mg.

### Hydrogel Formation

Lyophilised ECM tissue was digested enzymatically with pepsin, an endopeptidase found in the digestive system. Pepsin was added to 0.01 M HCl to create a 10% w/v solution, to which enough ECM tissue was added to create a 10 mg/mL stock digest solution. This digestion solution underwent constant agitation at 300 rpm for 48 hours at room temperature to achieve partial digestion. The solution was thereafter centrifuged and undigested material removed. The samples were then stored frozen at −20° C. until use.

Neutralisation of the acidic digest inactivated pepsin, allowing for collagen fibrillogenesis and eventual gelation. A neutralisation buffer of 8.89 mL 10×PBS, 3.11 mL 1×PBS and 8 mL of NaOH was created. The 10 mg/mL stock ECM digest was diluted and neutralised using 200 μL of buffer for every 800 μL of digest, yielding a neutralised solution at 8 mg/mL ECM concentration. This was mixed and pipetted into a 22 mm diameter brass olive ring to mould the gel into cylindrical shapes of 1.3 mL volume. The gelation of neutralised ECM digest is thermoresponsive, so samples were incubated at 37° C. for 40 minutes. Following complete gelation, ECM hydrogels were removed from the olive ring moulds. Samples were immediately cross-linked before mechanical assessment.

Cross-Linking with PA

PA was sourced from grape seed extract with a proanthocyanidin content of >95% (Bulkpowders, Sports Supplements Ltd, Colchester, UK). Dimethylsulphoxide (DMSO) was first added to the appropriate weight of PA so that the concentration of DMSO was 20% v/v in the final solution. Once fully suspended, PBS was added to create the final solution with the desired concentration of PA.

Crosslinking solution at the desired concentration was added to the bone tissue hydrogels at 4 times the volume of the hydrogel. The samples were placed on an orbital rocker at 150 rpm for 2 hours, before removing the crosslinking solution, washing twice with 1×PBS, then twice with dH₂O for 30 minutes each. Samples were either tested or processed immediately following crosslinking.

### Rheological Data Collection

Gels underwent rheological testing immediately following cross-linking using the Physica MCR 301 rheometer (Anton Paar, Hertford, UK) and a 25 mm parallel plate geometry. Gels were removed from well plates and transferred to peltier plate by spatula, ensuring the gel was supported and not overly manipulated. Gels were tested at 37° C. with a measurement gap determined by the amount of force being felt by the top parallel plate (generally from 2.7 mm-3.4 mm with a force of approximately 0.2 N). Amplitude sweep testing was conducted in the range of 0.1-200% strain at a constant angular frequency of 1 rad/s.

### Example 2—Preparation of a PA Cross-Linked Decellularized Liver Tissue Hydrogel of the Invention

A second embodiment of a PA cross-linked decellularized tissue hydrogel according to the invention was prepared by the following process.

### Tissue Processing and Decellularization

Fresh porcine liver was obtained through an EU certified butcher (R.B. Elliott & Son, Calow, Chesterfield, UK). This was supplied as a whole organ.

Liver tissue was decellularized without any prior processing steps.

Liver tissue was decellularized through an enzymatic and detergent based protocol, outlined by Loneker et al (A. E. Loneker, D. M. Faulk, G. S. Hussey, A. D'Amore, S. F. Badylak, Solubilized liver extracellular matrix maintains primary rat hepatocyte phenotype in-vitro, Journal of Biomedical Materials Research Part A 104(4) (2016) 957-965) and adapted in house. Liver tissue was shredded into 2.5-3.5 mm strips using a mandolin slicer, then washed three times in dH₂O for 30 minutes each. The following enzymatic and detergent based steps were performed at room temperature for 1 hour each at 300 rpm, with a tissue:volume ratio of 40 g tissue per 1.5 L of solution. A 0.02% trypsin/0.05% EDTA solution was used to facilitate dissociation of cells from the tissue, 3% (v/v) Triton-X-100 solutions were used to disrupt cell membranes and allow permeabilization, with a final 4% (w/v) sodium deoxycholate solution used to facilitate cell lysis via disruption of protein-protein interactions and solubilise cell membrane components. Between each of these processing steps, the tissue was separated using a 250 μm sieve and underwent three 15-minute wash steps with dH₂O. The tissue was also massaged in the sieve between each wash to help facilitate permeation of the detergents into the tissue. Resulting ECM material was stored in small volumes of dH₂O at 4° C. overnight before undergoing depyrogenation.

A solution of 0.1% peroxyacetic acid (PAA) diluted in 4% (v/v) ethanol and dH₂O was used as a depyrogenating wash for 2 hours at 300 rpm and room temperature. The PAA solution was used at a ratio of 1:20 (w/v) ECM:PAA solution. Following PAA treatment, four wash stages (2×PBS followed by 2×dH₂O) were performed for 15-minutes each at room temperature and 300 rpm, with resultant ECM lyophilised and stored at −20° C. until use.

### Hydrogel Formation

Hydrogel formation was performed in accordance with the method described in Example 1 for decellularized bone tissue.

Cross-Linking with PA

Cross-linking with PA was performed in accordance with the method described in Example 1 for decellularized bone tissue.

### Rheological Data Collection

Rheological data collection was performed in accordance with the method described in Example 1 for decellularized bone tissue.

### Example 3—Preparation of a PA Cross-Linked Decellularized SIS Tissue Hydrogel of the Invention

A third embodiment of a PA cross-linked decellularized tissue hydrogel according to the invention was prepared by the following process.

### Tissue Processing and Decellularization

Fresh porcine small intestine was obtained through an EU certified butcher (R.B. Elliott & Son, Calow, Chesterfield, UK). This was supplied as a whole organ.

Small intestine underwent a mechanically based decellularization technique, relying on a physical delamination of the tissue layers to leave behind the submucosal layer. Tissue was thoroughly washed through with tap water to remove tissue contents before being split open with a scalpel and forceps. The luminal side of the intestine was then scraped and delaminated using an acrylic or rubber-based scraper. The intestine was then turned over to scrape away the muscle and mucosal layers of the abluminal side. This process yielded the small SIS which was then washed under running dH₂O and stored at 4° C. until depyrogenation the following day. Depyrogenation of the tissue was performed with the same solutions and conditions used in liver decellularization in Example 2 before lyophilisation and storage.

### Hydrogel Formation

Hydrogel formation was performed in accordance with the method described in Example 1 for decellularized bone tissue.

Cross-Linking with PA

Cross-linking with PA was performed in accordance with the method described in Example 1 for decellularized bone tissue.

### Rheological Data Collection

Rheological data collection was performed in accordance with the method described in Example 1 for decellularized bone tissue.

### Results

FIGS. 1A and 1B show non cross-linked decellularized bone tissue hydrogels. FIG. 1A shows the decellularized bone tissue hydrogel as formed and FIG. 1B shows the decellularized bone tissue hydrogel when compressed.

The non cross-linked decellularized bone tissue hydrogels can withstand some manual handling such as removal from the well plate via spatula, however excessive force to, or manipulation of the gel would result in irreversible damage to the hydrogel. This is demonstrated in FIG. 1B when compression of the hydrogel is attempted.

FIG. 2 shows a GA cross-linked decellularized bone tissue hydrogel when compressed. The GA cross-linked tissue hydrogel largely retains its shape upon compression but is not able to be handled further.

FIG. 3A to 3D show decellularized bone tissue hydrogels cross-linked with PA.

FIGS. 3A and 3B show a PA cross-linked decellularized bone tissue hydrogel as formed and FIGS. 3C and 3D show a PA cross-linked decellularized bone tissue hydrogel when compressed into a flat sheet. PA cross-linking of bone tissue hydrogels retains the ECM gel structures and shapes. PA cross-linked gels are compressible into flat sheets without displaying structural weakness.

FIGS. 4A to 4D show PA cross-linked decellularized bone tissue hydrogels being rolled into hollow tubes. FIG. 4A shows a PA cross-linked decellularized bone tissue hydrogel in a curled formation, FIGS. 4B and 4C show the PA cross-linked decellularized bone tissue hydrogel loosely rolled to form a hollow tube and FIG. 4D shows the PA cross-linked decellularized bone tissue hydrogel tightly rolled to form another hollow tube. PA cross-linked tissue hydrogels are rollable into various structural forms. PA cross-linked bone tissue hydrogel does not display structural weakness even when tightly rolled into a tube as depicted in FIG. 4D.

FIG. 5 provides an overview of strength increases upon cross-linking of a decellularized bone tissue hydrogel with PA relative to a non cross-linked decellularized bone tissue hydrogel (NC); and compared with strength increases achieved by cross-linking identical decellularized bone tissue hydrogels with the identical corresponding concentrations of GA and GP. FIGS. 6A and 6B show storage moduli of non cross-linked (NC), GA cross-linked, GP cross-linked, and PA cross-linked decellularized bone tissue hydrogels. FIG. 6A shows storage moduli achieved with 15 minutes cross-linking and FIG. 6B shows storage moduli achieved with 2 hours cross-linking.

Cross-linking with PA confers the greatest increase in mechanical strength compared with the other cross-linking agents trialled (GA and GP). Although cross-linking with GP confers a small increase in mechanical strength, this is reduced compared to GA and PA cross-linking.

Increasing the cross-linking time and the concentration of the cross-linking agent employed had a more significant effect in increasing hydrogel strength for cross-linking with PA than with GA and GP. Notably, decellularized bone tissue hydrogel cross-linked with a 0.625% concentration of PA for 2 hours showed a large 77× strength fold increase compared to the non cross-linked equivalent. The storage moduli achieved with 2 hours of cross-linking with PA are more than adequate for bone repair. Generally, such high strength would result in brittle behaviour of a material, but the PA cross-linked hydrogel retains flexibility and compressibility, and remains a biocompatible and non-toxic biomaterial.

This behaviour was further indicated in yield stress experiments displayed in FIGS. 10A and 10B, in which tissue hydrogels crosslinked with PA showed average increases in yield stress up to 30-times that of the non-crosslinked hydrogel. Whereas GA and GP crosslinked hydrogels saw respective increases of only 5- and 3-times the non-crosslinked value.

As further shown by the storage moduli results in FIGS. 10A and 10B, increasing the concentration of PA crosslinker used from 10 mM to 50 mM provided crosslinked tissue hydrogels having storage moduli over twice as high, reaching values that were over 87-times the moduli of non-crosslinked gels. These results further demonstrate the flexibility of PA crosslinking and the fact that methods can be tailored based on the desired application to provide crosslinked tissue hydrogels of a vast spectrum of strengths and mechanical properties.

FIGS. 7A-7E and 8A-8E show PA cross-linked decellularized tissue hydrogels prepared from liver and SIS tissue, respectively. Hydrogels prepared from these tissue materials can similarly be compressed and rolled. Compressed/rolled hydrogels can be preserved by lyophilisation and rehydrated to give gels which largely maintain their previous shape. However, the hydrogels prepared from bone tissue generally display much greater strength and retain shape better when structurally manipulated.

Despite the higher strength of crosslinked tissue hydrogels prepared from bone tissue, all three of bone, liver and intestinal tissue display high natural strength, as demonstrated in Table 1 below. Table 1 shows storage moduli values of ECM hydrogels from different tissues pre-crosslinking.

TABLE 1

| ECM hydrogel tissue (8 mg/ml) | Storage modulus (Pa) |
|---|---|
| Bone | 509 ± 39 |
| SIS | 421 ± 27 |
| Liver | 515 ± 38 |
| Urinary bladder | 83 ± 12 |
| Spinal cord | 277 ± 12 |

The results show that bone, liver and SIS are particularly strong base tissues with higher natural strengths than bladder and spinal cord tissue. The natural tissue strength of such tissues combined with the reinforcement provided to the ECM structure on PA crosslinking thus allows for the provision of crosslinked tissue hydrogels with optimal mechanical properties.

Sulphated Glycosaminoglycan (sGAG) Content in Tissue Hydrogels

To further demonstrate the effect of decellularization and gelation on native ECM components, the sGAG contents of non-crosslinked bone, liver and SIS ECM tissue hydrogels were determined.

Results are displayed in FIG. 9 and show that sGAG contents post-decellularization and-gelation are largely similar to those of the native tissues. These results highlight the ability of such source tissues to maintain ECM structure and properties post-decellularization and-gelation, which can influence features such as growth factor binding and cellular response.

### Solvent Effects

The effect of the solvent used for crosslinking decellularized tissue hydrogels with PA was assessed. Table 2 below shows how the Complex Modulus G* of crosslinked tissue hydrogels varies depending on the crosslinking solvent used.

TABLE 2

| | | | 5% | 5% | | 10% | 10% |
|---|---|---|---|---|---|---|---|
| | | | propylene | Pluronic | | propylene | Pluronic |
| | Non- | 5% DMSO; | carbonate; | F127; | 10% DMSO; | carbonate; | F127; |
| | crosslinked | 1 mM PA | 1 mM PA | 10 mM PA | 10 mM PA | 10 mM PA | 10 mM PA |
| G* | 947 ± | 4703 ± | 3173 ± | 1019 ± | 33067 ± | 28900 ± | 1049 ± |
| (Pa) | 63 | 250 | 1047 | 202 | 2854 | 15819 | 126 |

Modulus values for crosslinked tissue hydrogels were all higher than for the non-crosslinked sample, indicative of the increase in strength that results from PA crosslinking. However, runs performed in DMSO and propylene carbonate solvents displayed particularly high Modulus values and increasing the crosslinker concentration was especially effective at increasing the hydrogel strength with these solvents.

These results were somewhat surprising as non-crosslinked tissue hydrogels which were incubated in DMSO and propylene carbonate showed considerably lower Modulus values compared to analogous tissue hydrogels which were incubated in Pluronic F127. In some cases, incubation of non-crosslinked hydrogels with these solvents even resulted in a considerable reduction in Modulus.

The results therefore demonstrate a synergistic effect which arises from the use of PA crosslinking with such polar aprotic solvents. Strong crosslinked tissue hydrogels are achieved and modifying the concentrations used allows for rheological properties to be tailored depending on the desired final properties.

### Degradation Assessment

The resistance of PA crosslinked decellularized tissue hydrogels to degradation was assessed in the respective presence of collagenase enzymes and Hank's Balanced Salt Solution (HBSS) buffer over a period of 126 days.

In the presence of collagenase, non-crosslinked tissue hydrogels and hydrogels treated with solvents alone completely degraded after only 1 day. Conversely, PA crosslinked tissue hydrogels prepared using varying concentrations of PA and in the presence of either a DMSO or propylene carbonate solvent maintained their shape and minimally degraded over the course of the full 126 days.

In the presence of HBSS buffer, the PA crosslinked tissue hydrogels on day 126 appeared largely identical to those on day 0. Non-crosslinked samples and samples treated with solvent alone, on the other hand, had mostly degraded by day 21.

These results demonstrate that crosslinking decellularized tissue hydrogels not only increases hydrogel strength but also protects the hydrogels from degradation even in the presence of harsh collagenase enzymes, which act to break down collagen, the main component present within ECM hydrogels.

### Toxicity Tests

The cytotoxicity of PA crosslinked decellularized tissue hydrogels was assessed by an LDH assay in which hydrogels were incubated with SH-SY5Y (human neuroblastoma cell line).

Crosslinkers tested were:
PA [publicly available food supplement]
Purified oligomeric PA (OPA) [reference standard quality]
GA
GP

These were all tested in 1 and 10 mM concentrations, with the 1 mM in a solution containing 5% v/v of DMSO, and the 10 mM in a solution containing 10% v/v DMSO.

The LDH assay quantifies how many cells have died by quantifying the level of lactate that is present in the culture medium which will have been released upon cell membrane disruption during their death. The assay contains the enzyme lactate dehydrogenase (LDH) which upon reacting with lactate releases NADH which in turn can react with the dye present in the assay solutions to produce a colorimetric change. This change can be read through absorbance on a microplate spectrophotometer.

All results for the LDH have been calculated to % cytotoxicity as per the following formula recommended by the manufacturer:

$$\% cytotoxicity = [ \frac{Compound treated LDH activity - Spontaneous LDH activity}{Maximum LDH activity - Spontaneous LDH activity} ] \times 100$$

Compound treated LDH activity=Sample of interest

Spontaneous LDH activity=Media only cells (negative control)

Maximum LDH activity=5% Triton-X-100 exposed cells (positive control)

### Indirect (Extract) Testing

Gels were made and crosslinked as per normal procedure and immersed in culture media for 72 hours conditioning. The conditioned culture media was then filtered and placed onto cells for a period of 24 hours. Following exposure to the conditioned media, a sample of the media was removed for testing through LDH.

Results are displayed in Table 3 below.

TABLE 3

| | LDH (% cytotoxicity relative to |
|---|---|
| | Triton) |
| Media | 0 |
| Non-crosslinked | −4.9 |
| 5% DMSO solvent (no crosslinker) | −4.5 |
| 10% DMSO solvent (no crosslinker) | −4.8 |
| PA (1 mM) | −4 |
| PA (10 mM) | 3.2 |
| OPA (1 mM) | −4.3 |
| OPA (10 mM) | 0.044 |
| GA (1 mM) | −3.1 |
| GA (10 mM) | −4.3 |
| GP (1 mM) | −4.2 |
| GP (10 mM) | 1.5 |
| 5% Triton | 100 |

All sample conditions showed a strong significant difference compared to Triton in the LDH assay. The data thus illustrates that the eluted products of PA crosslinkers have no significant standing on cytotoxicity.

### Direct Testing

Gels were made and crosslinked as per normal procedure and immersed in Hanks balanced salt solution (HBSS) containing 1% v/v of antibiotics and antimycotics overnight. The gels were then removed and immersed into culture media for 2 hours before placing on top of the cell monolayer and ensuring direct contact. The cells were in contact and cultured in the presence of the gel for 24 hours before a sample of the media was removed for testing through LDH.

Results are displayed in Table 4 below.

TABLE 4

| | LDH (% cytotoxicity relative to |
|---|---|
| | Triton) |
| Media | 0 |
| Non-crosslinked | −4.5 |
| 5% DMSO solvent (no crosslinker) | 5.6 |
| 10% DMSO solvent (no crosslinker) | −0.7 |
| PA (1 mM) | 6 |
| PA (10 mM) | 5.9 |
| OPA (1 mM) | 2 |
| OPA (10 mM) | 6.7 |
| GA (1 mM) | 0.3 |
| GA (10 mM) | 2 |
| GP (1 mM) | −1.5 |
| GP (10 mM) | 2.8 |
| 5% Triton | 100 |

Once again, all sample conditions saw a significant decrease in LDH when compared to Triton.

Overall, no large variations in relative LDH levels are seen between non-crosslinked tissue hydrogels and tissue hydrogels cross-linked with different concentrations of PA.

No significant increases in cytotoxicity were found when crosslinking was performed using PA versus GA and GP crosslinking. All PA runs varied minimally compared to the media control and were within acceptable cytotoxicity ranges.

Overall, the results demonstrate that crosslinking decellularized tissue hydrogels with PA has minimal effects on cytotoxicity for both extract testing and direct testing, irrespective of the PA concentration used.

Crosslinking with Tea

Catechins and other polyphenols are present in tea which are functionally and structurally similar to components present in PA. Crosslinking of decellularized tissue hydrogels using tea was thus tested and performance evaluated.

Black tea (Twinings Everyday Blend) was steeped in boiling water for 1 hour, after which the tea bag was removed, and the tea solution allowed to cool for a further 1hour. No other solvents were present in the tea solution. Decellularized tissue hydrogels were prepared as per the protocols described above and were thereafter submerged in the prepared tea solution for 2 hours at room temperature as per the standard crosslinking procedure described above.

Crosslinked gels were flexible and compressible. The strength of the crosslinked gel was assessed by calculation of its storage modulus and comparison with a non-crosslinked gel (NC). Results are displayed in Table 5 below.

TABLE 5

| | Storage Modulus, G′ (Pa) | Fold change to NC |
|---|---|---|
| NC | 409 ± 42 | N/A |
| Tea | 3023 ± 931 | 7.4 |

A significant 7.4-fold increase was achieved on tea crosslinking compared to non-crosslinked gels. Surprisingly, such results were achieved without assistance from polar aprotic solvents and using only the relatively low natural polyphenol concentration present in tea.

Crosslinking with Curcumin

Crosslinking of decellularized tissue hydrogels using polyphenols in curcumin was also investigated.

A 50 mM curcumin solution in 100% DMSO was prepared and a decellularized tissue hydrogel, prepared according to the above protocols, was submerged in the solution for 2 hours as per the standard crosslinking procedure described above.

The strength of the crosslinked gel was assessed by calculation of its storage modulus 5 and comparison with a non-crosslinked gel (NC). Results are displayed in Table 6 below.

TABLE 6

| | Storage Modulus, G′ (Pa) | Fold change to NC |
|---|---|---|
| NC | 296 | N/A |
| Curcumin 50 mM in | 1610 | 5.4 |
| 100% DMSO | | |

A significant 5.4-fold increase was achieved on curcumin crosslinking compared to the non-crosslinked gel.

The above embodiments are described by way of example only. Many variations are possible without departing from the scope of the invention as defined in the appended claims.

1. A decellularized tissue hydrogel cross-linked with a polyphenol.

2. A decellularized tissue hydrogel as claimed in claim 1, wherein the decellularized tissue hydrogel comprises one or more tissues selected from the group comprising bone, liver, intestine, and amniotic membrane.

3. A decellularized tissue hydrogel as claimed in claim 2, wherein the bone tissue comprises one or more tissue types selected from the group comprising compact tissue, cancellous tissue, subchondral tissue and combinations thereof.

4. A decellularized tissue hydrogel as claimed in any preceding claim, wherein the polyphenol comprises one or more compounds selected from the group comprising a flavonoid, a hydroxybenzoic acid, a polyphenolic amide, a hydroxycinnamic acid, a stilbene, a lignan and combinations thereof.

5. A decellularized tissue hydrogel as claimed in claim 4, wherein the polyphenol comprises a flavonoid comprising a condensed tannin.

6. A decellularized tissue hydrogel as claimed in claim 5, wherein the polyphenol comprises a condensed tannin comprising a proanthocyanidin or any glycoside thereof.

7. A decellularized tissue hydrogel as claimed in claim 6, wherein the polyphenol comprises a proanthocyanidin comprising one or more compounds selected from the group comprising a procyanidin, a propelargonidin, a prodelphinidin, a profisetinidin, a proteracacinidin, a proguibourtinidin, a prorobinetidin, a propetunidin, a promalvidin, a propeonidin and combinations thereof.

8. A decellularized tissue hydrogel as claimed in any preceding claim, wherein the polyphenol is of plant origin, which may comprise fruit, nut, vegetable or spice.

9. A decellularized tissue hydrogel as claimed in claim 8, wherein the polyphenol is of tea origin.

10. A decellularized tissue hydrogel as claimed in claim 8, wherein the polyphenol is of grape origin, preferably of grape seed or grape skin origin.

11. A pharmaceutical composition comprising a decellularized tissue hydrogel cross-linked with a polyphenol.

12. A method of preparing a cross-linked decellularized tissue hydrogel, the method comprising the steps of:
a. Providing at least one decellularized tissue hydrogel; and
b. Cross-linking the at least one decellularized tissue hydrogel with a polyphenol.

13. A method of preparing a cross-linked decellularized tissue hydrogel as claimed in claim 12, wherein step (a) comprises preparing the at least one decellularized tissue hydrogel from bone tissue by a method comprising the steps of: optionally demineralising and delipidating the bone tissue, decellularizing the bone tissue, digesting the decellularized bone tissue with at least one enzyme to form a digest, and neutralising the digest.

14. A method of preparing a cross-linked decellularized tissue hydrogel as claimed in claim 13, wherein the step of demineralising and delipidating the bone tissue comprises subjecting the bone tissue to hydrochloric acid and subjecting the bone tissue to a mixture of chloroform and methanol.

15. A method of preparing a cross-linked decellularized tissue hydrogel as claimed in any one of claims 12 to 14, wherein step (b) comprises cross-linking the at least one decellularized tissue hydrogel with a polyphenol for at least 2 hours.

16. A method of preparing a cross-linked decellularized tissue hydrogel as claimed in any one of claims 12 to 15, wherein step (b) further comprises subjecting the at least one decellularized tissue hydrogel to agitation.

17. A method of preparing a cross-linked decellularized tissue hydrogel as claimed in any one of claims 12 to 16, wherein the polyphenol has a concentration of at least 0.1 mM.

18. A decellularized tissue hydrogel cross-linked with a polyphenol for use in the treatment of a wound, condition or injury.

19. A decellularized tissue hydrogel cross-linked with a polyphenol as claimed in claim 18, wherein the wound, condition or injury comprises a wound, condition or injury to bone, nerves, tendons or abdominal regions
